(19) United States
(12) Patent Application Publication
(10) Pub. No.: US 2001/0034545 A1
(43) Pub. Date: Oct. 25, 2001

(54) Conformal therapy component of an animate body heat exchanger having adjustable length tongue

(76) Inventor: William Elkins, San Jose, CA (US)

Correspondence Address: MICHAEL B. EINSCHLAG, ESQ., ROSENLAW.COM, 702 MARSHALL STREET, SUITE 301, REDWOOD, CA, 94063, US

(21) Appl. No.: 09/771,125

(22) Filed: Jan. 26, 2001

Related U.S. Application Data
(60) Provisional application No. 60/178,983, filed on Jan. 28, 2000.

(51) Int. Cl. A61F 7/00

(52) U.S. Cl. 607/108

(57) ABSTRACT

A conformable therapy component of an animate body heat exchanger is described, which component is particularly designed for the back of a human. The component includes two registering compliant bladders for (1) confining a flowing liquid heat exchange medium, and (2) providing air pressure. It also includes a flexible tongue and a metal loop which interacts with such tongue to hold the operational aspects of the component in place conforming closely to the back of a therapy patient.

### CROSS REFERENCE TO RELATED APPLICATIONS

Some of the subject matter described and claimed in this patent application is also disclosed in provisional patent application Ser. No. 60/178,983 filed Jan. 28, 2000, the priority of which is hereby claimed.

### DISCLOSURE

Background of the Invention

The present invention relates to an animate body heat exchanger and, more particularly, to a conformal heat exchange therapy component for a desired interaction with a part of an animate body to be subjected to therapy, which component includes a securance structure for holding the active part of the same in a desired position relative to the body part.

It is now common to apply cold and compression to a traumatized area of a human body to facilitate healing and to prevent unwanted consequences of the trauma. In fact, the acronym RICE (Rice, Ice, Compression, and Elevation) is now used by many.

Cold packing with ice bags or the like has traditionally been used to provide deep core cooling of a body part. Elastic wraps are often applied Lo provide compression.

It will be appreciated that the traditional techniques are quite uncontrollable. For example, the temperature of an ice pack will, of course, change when the ice melts; and it has been shown that the application of elastic wraps and, consequently, the pressure provided by the same, varies considerably even when the wrapping is done by an experienced individual.

Because of these and other difficulties, many in the field have turned to more complicated animate body heat exchangers. Most effective animate body heat exchangers typically include two major components—an external conformal therapy component for covering a body part to be subjected to heat exchange and compression, and a control component for producing a flowing heat exchange fluid and a pressurized gas. The external conformal therapy component typically includes two bladders, a first one of which confines a heat exchange liquid or other medium and a second of which overlays the first and confines gas pressure to be applied to the body part to inhibit edema and to apply pressure against the heat exchange liquid bladder to press it toward the body part.

One problem faced by makers of heat exchange therapy components is that it is difficult to design the same in a way which will assure that it does not shift—it remains conformed to the body part to be treated during the application of a heat exchange liquid and gas pressure. This problem is compounded by variations in the individual sizes of body parts to be treated. This latter aspect is particularly troublesome if the therapy component is designed to treat body parts which naturally vary in size from individual to individual.

### SUMMARY OF THE INVENTION

Thee present invention addresses the above problem. A thermal therapy component according to the invention includes not only the regular bladder or bladders to provide a desired treatment, but also a special securance structure for holding the bladders in a desired position relative to the body part to be treated. This special securance structure includes a tongue designed to pass about an object which is essentially stationary relative to the body part.

The length of the tongue is adjustable, and the securance structure also includes a direction controller which is positioned to interact with the tongue and define its path in passing about the object. A position securance device is also included on the tongue to aid in holding it interacting with the direction controller. This interaction results in the tongue being folded back on itself.

In the preferred embodiment, it is the back of a human which is to be treated and the object around which the tongue passes is the torso of such human. The length of the tongue is itself adjustable by having the same made up of a pair of segments which are adjustable in position relative to one another lengthwise of the tongue. This adjustment is easily provided by making one of the segments a sleeve and telescoping the other within it. Moreover, the free end of the tongue includes a hook section of a Velcro™-type hook and fastener arrangement with the remainder of the therapy component (being loop fastening material) at the location of securance of the tongue free end.

Other features and advantages of the invention either will become apparent or will be described in connection with the following, more detailed description of a preferred embodiment of the invention and variations.

### BRIEF DESCRIPTION OF THE DRAWING

With reference to the accompanying sheets of drawing:

FIGS. 1A and 1B are isometric views of a human being treated with a preferred embodiment of the invention, the control unit of a complete animate body heat exchanger;

FIG. 2 is a broken away elevation view of the preferred embodiment of he invention;

FIG. 3 is a partial elevation view of an alternate arrangement obtainable With the preferred embodiment of the invention; and

FIG. 4 is an enlarged partial sectional view showing some details of such preferred embodiment.

### DETAILED DESCRIPTION OF THE PREFERRED EMBODIMENT

The following, relatively detailed description is provided to satisfy the patent statutes. It will be appreciated by those skilled in the art, though, that various changes and modifications can be made without departing from the invention.

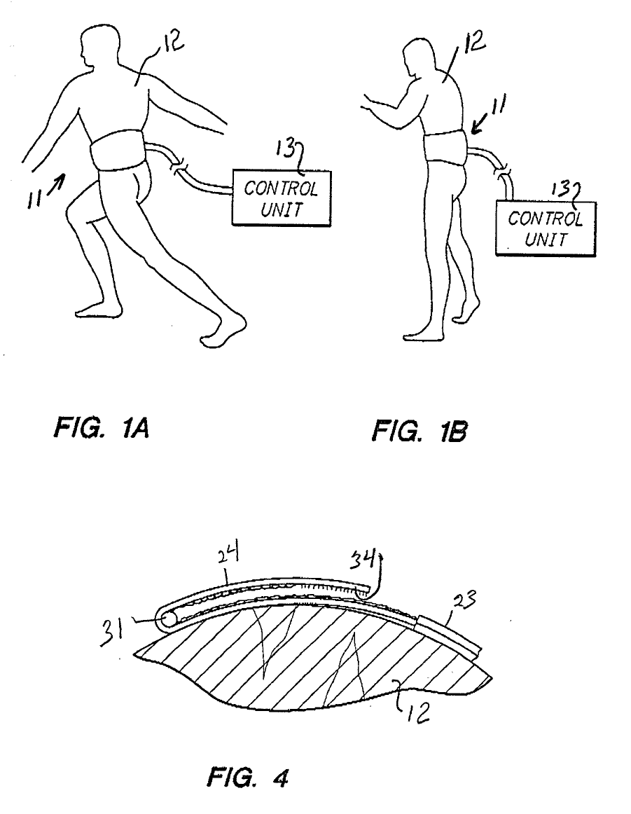

The conformal therapy component for the back of the human is generally referred to in FIGS. 1A and 1B by the reference numeral 11. As is illustrated, the component 11 encircles the torso of a human 12 (the torso is essentially stationary relative to the back) to hold the operational aspects of the component in place, and a control unit for the component is represented at 13.

It is important for a conformal therapy component to be effective, that it be quite closely associated with the body part to be treated. However, the torsos of individual humans (especially at the waist lines) vary in size greatly. The conformal therapy component of the invention is particularly designed, as will be described in more detail hereinafter, to accommodate these variations and provide the close conformance of its operating aspects that is desired. One of such aspects is a compliant bladder for confining a heat exchange medium, such as a liquid.

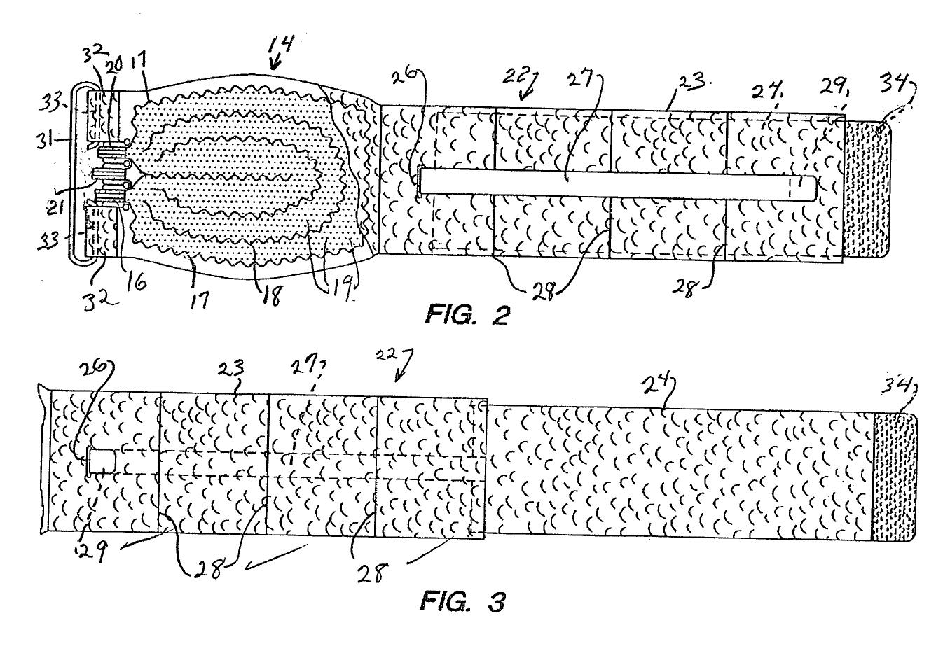

The interior layout of the above bladder is shown in FIG. 2 and is referred to therein by the reference numeral 14. The flow of heat exchange liquid into the same is via a tube 16. Its flow throughout the bladder is confined and guided by a curvilinear border 17 and curvilinear fences or dividers 18. In accordance with Patent application Ser. No. 09/173,637 filed originally Jul. 21, 1998 and naming the inventor hereof as a co-inventor, the curvilinear border 17 and the fences 18 are formed by a plurality of curvilinear ripples which generally are significantly shorter than the length of the border or fence, respectively, of which each is a part. This curvilinear ripple construction, among other things, prevents eddies from forming during liquid flow through the bladder. A matrix or, in other words, a pattern of dots 19 are provided throughout the interior of the bladder to disperse the flowing liquid supplied by the control unit. Preferably, such pattern conforms to that described in U.S. patent application Ser. No. 09/127,256 originally filed Jul. 31, 1998 and naming the instant inventor as the inventor. The liquid is circulated back to the control unit by way of a tube 20. Constant circulation through the bladder of a controlled temperature liquid via tubes 16 and 20 assures chat a controlled temperature is maintained in the bladder. In a major realization of the preferred embodiment, the liquid was maintained at a temperature of about 45° F., a temperature which is cold relative to a normal, live human body. In this connection, the control unit included an ice bath surrounding a container through which the liquid was circulated after being returned from the heat exchange bladder. (The control used in this implementation was capable of supplying liquid at other controlled temperatures.) in the realization discussed above, the liquid was a 20 percent propylene glycol solution in distilled water with a small amount of a wetting agent to break surface tension, and iodine as an anti-fungicide. The control unit included an ice bath surrounding a container through which the liquid was circulated after being returned from the heat exchange bladder 14. The control unit used in this realization was capable of supplying liquid at other controlled temperatures.

The bladder is defined by flexible walls and is configured to be positioned in a thermally conductive relationship with the back of the Individual who is wearing the conformal therapy component. The result is that the flowing liquid absorbs thermal energy from the human back. Such heat exchange bladder is defined by two flexible sheets of material which are heat sealed to one another to form the border 17. One of these sheets is behind the one shown and registers with it. Such hidden sheet forms one wall of a compliant gas pressure bladder which overlays the heat exchange liquid bladder. This gas pressure bladder is also defined by an exterior sheet of material forming an exterior wall of the same and playing a major part an defining the securance structure to be described in more detail below. The exterior surface of this sheet of material is provided with Velcro™-type loop material both for aesthetic reasons and for securance reasons, as will be discussed below.

The gas pressure bladder registers with the heat exchange liquid bladder and, in this connection, its outer walls are also defined by the heat sealing process which provides the border 17 for the liquid bladder.

The sheets of material providing the two bladders and most of the securance structure to be defined below are nylon coated with a polyethylene to provide the same with both with the heat sealing qualities and impermeability to liquid and air as appropriate.

The interior of the gas pressure bladder is the same as the interior of the liquid bladder, except that the dots 19 are not provided. The purpose of the fences in such bladder are to maintain the bladder walls adjacent one another at various locations and, thus, prevent “ballooning.” (It will be understood that in some embodiments it may not be desirable to provide the fences within the gas pressure bladder.)

Gas pressure (typically air pressure) is introduced into he gas pressure bladder via tube 21. In the realization mentioned above, a cyclic air pressure, cycling between 0.25 psig and 1.5 psig every two minutes from peak to peak was introduced into the pressure bladder.

In keeping with the invention, the outer sheet of material forming the external wall of the gas pressure bladder is extended to aid in forming the previously mentioned securance structure. More particularly, it forms a tongue 22 which projects from the portion of the therapy component having the compliant bladders. As illustrated, such tongue has a length in the direction of such projection, which length is adjustable. That is, with reference to FIGS. 2 and 3 is seen that the tongue 22 is made up of a sleeve segment 23 within which a segment 24 is telescoped. Thus, these segments are adjustable in position relative to one another to thereby adjust the length of the tongue.

To facilitate such adjustment, segment sleeve 23 includes a slot 26 adjacent the bladder through which a strap 27 projects. One end of the strap 27 is secured via sewing or the like to the segment 24 adjacent the end of the same which is interiorly of segment 23. Such strap is folded back at slot 26 and projects Lengthwise along segment 23. The interior surface of segments 23 and 24 is formed by Velcro™-type loop material, and segment 23 includes indicia (lines 28) extending laterally thereof indicating three different spaced positions, which indicia will be discussed in more detail below. The free end of strap 27 includes a pad 29 of Velcro™-type hook fastening material to thereby enable such strap to be secured to segment 23.

It will be seen by comparing FIGS. 2 and 3 how the length of the tongue is adjustable via the telescoping segments 23 and 24 and the strap 27. For inward telescoping, the strap 27 only needs to be pulled outward through slot 26. For outward telescoping the segment 24 can be pulled outward from segment 23. The free end of the strap facilitates determining a selected length adjustment. That is, the free end of the strap is made to line up with one of the indicia lines 28 and then secured to the loop material of the segment 23. These lines are positioned along the length of sleeve 23 as shown so that when the strap free end is secured at one of the same, that one will indicate the total length of the tongue provided by the two segments. It will be appreciated that other or more indicia can be provided.

A conformal therapy component incorporating the invention also includes a direction controller positioned to interact with the tongue and define its path. In this preferred embodiment, such direction controller takes the form of a metal loop 31 which is secured to the end of the therapy component opposite that from which the tongue projects, by a pair of projecting tabs 32 (see FIG. 2) provided by the material forming the outer wall of the air bladder. As shown, the tabs 32 encircle corresponding re-entrant hook portions 33 of the hook 31.

In use, the operative aspect of the therapy component are positioned at the wearer's back, and the tongue 22 is passed about the torso of such wearer and through the loop 31 so that the component can be cinched relatively tightly in position. Such tongue is folded back on itself after passing through the loop 31, and its free end is secured to the remainder of the therapy component. In this connection, a pad 34 of a Velcro-type hook material is provided on the free end of the segment 24 and, hence, the tongue is securable to the exterior surface of the remainder of the therapy component. In this connection, it must be remembered that the exterior surface of the tongue 24 is loop material. (The inner surface of surface of the tongue is also provided with the loop material for aesthetic purposes.) Thus, it will be seen that with this type of position securance, the operational aspects of the therapy component easily can be positioned and then because of the cinching operation will be held in good thermally conductive relationship with the human's back.

As mentioned at the beginning of the detailed description, applicant is not limited to the specific embodiment and variations described above. They are exemplary, rather than exhaustive. The claims, their equivalents and their equivalent language define the scope of protection.

1. A conformal therapy component of an animate body heat exchanger, which therapy component is designed to apply heat exchange to an animate body part, comprising:
a. a compliant bladder for confining a heat exchange medium, which bladder is configured to be positioned in a thermally conductive relationship with said body part; and
b. a securance structure for holding said bladder in a desired position relative to said body part, said securance structure including:
(1) a tongue for passing about an object which is essentially stationary relative to said body part, said tongue projecting from said portion of said therapy component having said compliant bladder and having a length in the direction of such projection;
(2) a direction controller positioned on said therapy component to interact with said tongue and define its path passing about said object; and
(3) a position securance device on said tongue to aid in holding said tongue interacting with said direction controller passed about said object.

2. The conformal therapy component of claim 1 wherein the length of said tongue is adjustable.

3. The conformal therapy component of claim 2 further including a compliant gas pressure bladder overlaying a least a portion of the heat exchange medium bladder to inhibit edema of said animate body part at the location of said gas pressure bladder and to direct gas pressure against the heat exchange medium bladder to press the latter bladder toward said body part.

4. The conformal therapy component of claim 2 wherein said body part is a human back and said object is the torso of the human.

5. The conformal therapy component of claim 2 wherein said tongue has a pair of segments, a first one of which is adjustable in position relative to the second one to adjust the length of said tongue.

6. The conformal therapy component of claim 5 in which one of said segments includes indicia which indicates the total length of said tongue provided by said two segments when said segments are associated with one another at selected indicia.

7. The conformal therapy component of claim 5 wherein one of said segments is a sleeve and the other is telescoped within the same to facilitate such adjustment in relative position.

8. The conformal therapy component of claim 2 wherein said tongue is flexible and interacts with and passes through said direction controller to be folded back on itself thereby to provide some adjustment in holding said tongue in place about said object, and said position securance device is on the free end of said tongue and cooperates with other aspects of said therapy component to provide said holding.

9. The conformal therapy component of claim 8 wherein said position securance device provided on the free end of said tongue is a hook or loop fastening mechanism, and the remainder of said component at the location at which the tongue free end is to be secured includes the other of said hook and loop fastening mechanism.

10. A conformal therapy component of an animate body heat exchanger, which therapy component is designed to apply heat exchange to an animate body part, comprising:
a. a compliant bladder for confining a heat exchange liquid, which bladder is configured to be positioned in a thermally conductive relationship with said body part;
b. a compliant gas pressure bladder overlaying at least a portion of the heat exchange fluid bladder to direct gas pressure against said animate body part to inhibit edema of said body part, and against the heat exchange liquid bladder to press the latter bladder toward said body part; and
c. a securance structure for holding said bladder in a desired position relative to said body part, said securance structure including:
(1) a flexible tongue for passing about an object which is essentially stationary relative to said body part, said tongue projecting from said portion of said therapy component having said bladders and having a length in the direction of such projection defined by a pair of segments which are adjustable in position relative to one another lengthwise of said tongue to thereby change the length of said tongue;
(2) a direction controller positioned to interact with said tongue defining its path passing about said object, said tongue being arranged to interact thigh said direction controller and to be folded back on itself; and
(3) a position securance device on the free end of said tongue to aid in holding said tongue interacting with said direction controller and passed about said object.

11. The conformal therapy component of claim 10 wherein said body part is a human back and said object is the torso of the human.

12. The conformal therapy component of claim 10 wherein said position securance device provided on the free end of said tongue is hook or loop fastening mechanism, and the remainder of said component at the location at which the tongue free end is to be secured includes the other of said hook and loop fastening mechanism.
